(19) United States
(12) Patent Application Publication
(10) Pub. No.: US 2015/0157824 A1
(43) Pub. Date: Jun. 11, 2015

(54) CUSHION WITH COMPRESSION CONTROL

(71) Applicant: KONINKLIJKE PHILIPS N.V., EINDHOVEN (NL)

(72) Inventor: Peter Chi Fai Ho, Pittsburgh, PA (US)

(21) Appl. No.: 14/418,317

(22) PCT Filed: Jul. 11, 2013
(86) PCT No.: PCT/IB2013/055705

§ 371 Date: Jan. 29, 2015

Related U.S. Application Data
(60) Provisional application No. 61/678,691, filed on Aug. 2, 2012.

(51) Int. Cl. A61M 16/06

(52) U.S. Cl. CPC A61M 16/0622

(57) ABSTRACT

A cushion (10) for use in a patient interface device includes a first portion (12) structured to be coupled to a frame of a patient interface, an opposite second portion (14) structured to sealingly engage a patient's face, and a wall portion (16) disposed therebetween. The wall portion includes a base portion (20) extending from the first portion and an angled portion (24) extending from the base portion at an angle (a) with respect to the base portion. The second portion includes an underlying support portion (26) which extends from about a mid-portion of the angled portion and a membrane (30) which extends generally from an end of the angled portion. The angled portion is structured to generally maintain the lateral positioning of the membrane and the support portion when the cushion is transitioned from an uncompressed position to a compressed position.

### CROSS-REFERENCE TO RELATED APPLICATIONS

This patent application claims the priority benefit under 35 U.S.C. §119(e) of U.S. Provisional Application No. 61/678,691, filed on Aug. 2, 2012, the contents of which are herein incorporated by reference.

### BACKGROUND OF THE INVENTION

1. Field of the Invention

The present invention generally relates to a cushion for a patient interface, and, more particularly, to a cushion for a patient interface in a pressure support system that supplies a flow of gas to the airway of a patient.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation (NIV). It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), chronic obstructive pulmonary disease (COPD), or congestive heart failure (CHF).

Non-invasive ventilation and pressure support therapies involve the placement of a patient interface device, which is typically a nasal or nasal/oral mask, on the face of a patient to interface the ventilator or pressure support system with the airway of the patient so that a flow of breathing gas can be delivered from the pressure/flow generating device to the airway of the patient.

Typically, patient interface devices include a mask shell or frame having a cushion attached to the shell that contacts the surface of the patient. The mask shell and cushion are held in place by a headgear that wraps around the head of the patient. The mask and headgear form the patient interface assembly. A typical headgear includes flexible, adjustable straps that extend from the mask to attach the mask to the patient.

Because such masks are typically worn for an extended period of time, a variety of concerns must be taken into consideration. For example, in providing CPAP to treat OSA, the patient normally wears the patient interface device all night long while he or she sleeps. One concern in such a situation is that the patient interface device is as comfortable as possible, otherwise the patient may avoid wearing the interface device, defeating the purpose of the prescribed pressure support therapy. It is also important that the interface device provide a tight enough seal against a patient's face without discomfort. A problem arises in that in order for the mask to maintain a seal without any undue gas leaks around the periphery of the mask, the mask may be compressed against the patient's face.

Conventional CPAP masks that seal by compression commonly cause uncomfortable pressure points and do not adjust well to different anatomical facial features. The conventional concept of a compression seal is to generally displace tissues on the patient's face in order to achieve a uniform seal. Tissue displacement results in pressure points, skin markings (i.e., red marks), indentations, and overall prolonged discomfort. Conventional seals also commonly distort outward upon compressing and in doing so tend to pull or push on portions of a patient's face in a manner which is uncomfortable and can commonly lead to unwanted leaks.

Accordingly, a need exists for a cushion for a patient interface device that improves upon existing devices, for example, to maximize patient comfort while minimizing leak, during delivery of a positive airway pressure or flow of gas to the airway of the user.

### SUMMARY OF THE INVENTION

As one aspect of the present invention a cushion for use in a patient interface device is provided. The cushion comprises: a first portion structured to be coupled to a frame of a patient interface, an opposite second portion structured to sealingly engage a patient's face, and a wall portion disposed between the first portion and the second portion. The wall portion comprises: a base portion extending from the first portion; and an angled portion extending from the base portion generally outward at an angle with respect to the base portion toward the second portion. The second portion includes: an underlying support portion which extends from about a mid-portion of the angled portion and a membrane which extends generally from an end of the angled portion opposite the base portion. The angled portion is structured to generally maintain the lateral positioning of the membrane and the support portion when the cushion is transitioned from an uncompressed state to a compressed state.

The angled portion may be generally separated into a first segment and a second segment by the support portion; the first segment extending from the base portion to the support portion and the second segment extending from the support portion to the membrane. The first segment may have a predetermined thickness T₁, the second segment may have a predetermined thickness T₂ and the base portion may have a predetermined thickness T₃. The predetermined thickness T₃ may be equal to the predetermined thickness T₂, which may be greater than the predetermined thickness T₁. The predetermined thickness T₃ may be greater than the predetermined thickness T₂, which may be greater than the predetermined thickness T₁. The predetermined thickness T₂ may be greater than the predetermined thickness T₃, which may be greater than the predetermined thickness T₁.

The angled portion may include a selectively placed groove structured to maintain lateral positioning of the membrane and the support portion when the cushion is transitioned from an uncompressed state to a compressed state. The groove may be disposed adjacent the support portion. The groove may be disposed adjacent the base portion.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are provided for the purpose of illustration and description only and are not intended as a definition of the limits of the invention.

### BRIEF DESCRIPTION OF THE DRAWINGS

FIG. 1 is an isometric view of the patient side of an example embodiment of a cushion for a patient interface device according to the principles of the present invention;

FIG. 2 is an elevation view of the patient facing side of the cushion of FIG. 1;

FIG. 3 is a cross-sectional view of the cushion of FIGS. 1 and 2 taken along line 3-3 of FIG. 2 showing the cushion in a relaxed, uncompressed position;

FIG. 4 is a version of the cross-sectional view of FIG. 3 showing the cushion in a compressed position, such as would occur when the cushion is pressed against the face of a patient;

FIG. 5 is a cross-sectional view of a portion of another example embodiment of a cushion according to the principles of the present invention shown in a relaxed, uncompressed position;

FIG. 6 is a version of the cross-sectional view of FIG. 5 showing the portion of the cushion in a compressed position, such as would occur when the cushion is pressed against the face of a patient;

FIG. 7 is a cross-sectional view of a portion of yet another example embodiment of a cushion according to the principles of the present invention shown in a relaxed, uncompressed position; and

FIG. 8 is a version of the cross-sectional view of FIG. 7 showing the portion of the cushion in a compressed position, such as would occur when the cushion is pressed against the face of a patient.

### DETAILED DESCRIPTION OF THE EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality) and the singular form of “a”, “an”, and “the” include plural referents unless the context clearly indicates otherwise.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

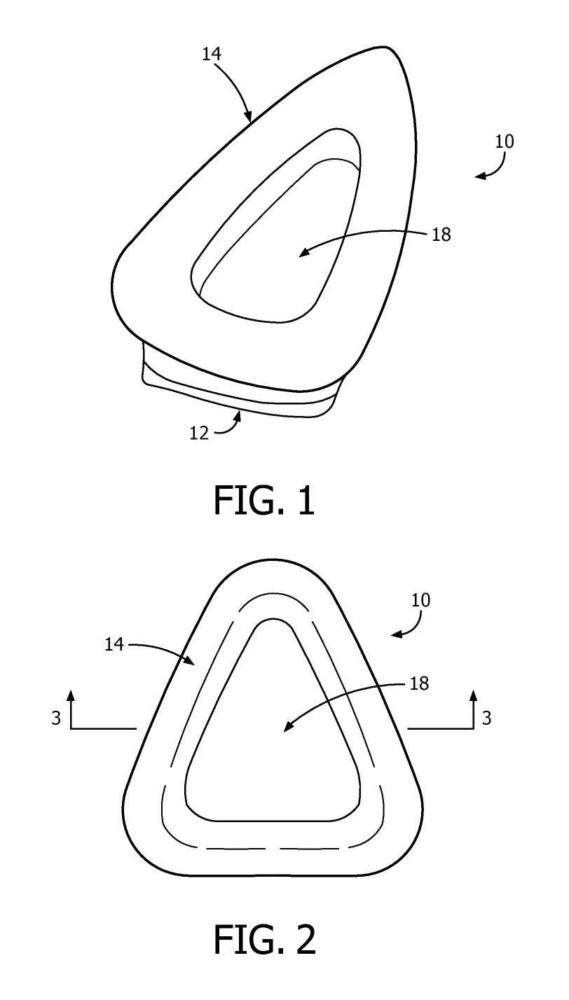

FIGS. 1-4 illustrate a cushion 10 constructed according to an example embodiment of the present invention. Cushion 10 is adapted to be removably or permanently connected (e.g., via mechanical and/or adhesive fastening) to a frame of a patient interface (not shown) structured to deliver breathable gas to a patient. In an embodiment, cushion 10 may be co-molded to a frame of a patient interface. In another embodiment, cushion 10 may form part of a frame with an outer support structure. Regardless of the particular application, cushion 10 provides a seal with the patient's face during use.

In example embodiments, cushion 10 is formed from a silicone rubber generally in the range of 20 to 50 Shore A. However, any soft rubber such as, for example, without limitation, TPE (thermal plastic elastomer) or any other suitable material may be employed without varying from the scope of the present invention.

In the embodiment illustrated in FIGS. 1-4, cushion 10 forms a part of a full-face mask. Specifically, cushion 10 provides a seal around the patient's nose and mouth to enable the delivery of breathable gas to the airway of a patient via the patient's nose and mouth. It is to be appreciated, however, that aspects of the present invention may be applicable to other breathing arrangements, e.g., without limitation, a nasal mask, an oral mask, etc. Cushion 10 is structured to provide a more comfortable fit for a wide range of facial shapes and sizes. Also, cushion 10 is structured to provide a better seal and reduce the risk of leakage as discussed below. Cushion 10 may be provided in various sizes in order to accommodate various facial sizes.

As illustrated in FIGS. 1-4, cushion 10 includes a first portion 12 structured to be connected to a frame (not shown) of a patient interface (e.g., without limitation, via a friction-fit, a tongue-and-groove arrangement, etc.), an opposite second portion 14 structured to sealingly engage the patient's face, and a wall portion 16 disposed between first portion 12 and second portion 14, generally forming a cavity 18 for communicating a flow of gas from a conduit coupled to the frame of the patient interface to the airway of the patient. As best shown in the elevation view of FIG. 2, second portion 14 has a generally triangular shape and is structured to continuously contact the nasal bridge, side of nose, upper cheek, lower cheek, and chin regions of the patient. However, second portion 14 may be formed in other suitable shapes, e.g., without limitation, a generally trapezoidal shape.

Referring to FIGS. 2-4, wall portion 16 of cushion 10 includes a base portion 20 which extends away from first portion 12. In the example embodiment illustrated in FIGS. 1-4, base portion 20 extends generally perpendicular to, and away from, an end face 22 of first portion 12. Wall portion 16 further includes an angled portion 24 extending from base portion 20 generally outward at an angle α with respect to base portion 20 toward second portion 14. In example embodiments of the invention, angled portion 24 is typically in the range of about 25° to about 45°, however other angles may be employed.

Second portion 14 of cushion 10 includes an underlying support portion 26, which extends from about a mid-portion of angled portion 24, and a membrane 30 which extends generally from an end of angled portion 24 opposite base portion 20. Support portion 26 adds rigidity to membrane 30 at the sides of the patient's mouth and cheeks. While it is preferable that membrane 30 be thinner than the underlying support portion 26, they could have the same thickness or membrane 30 could be thicker than the underlying support portion 26.

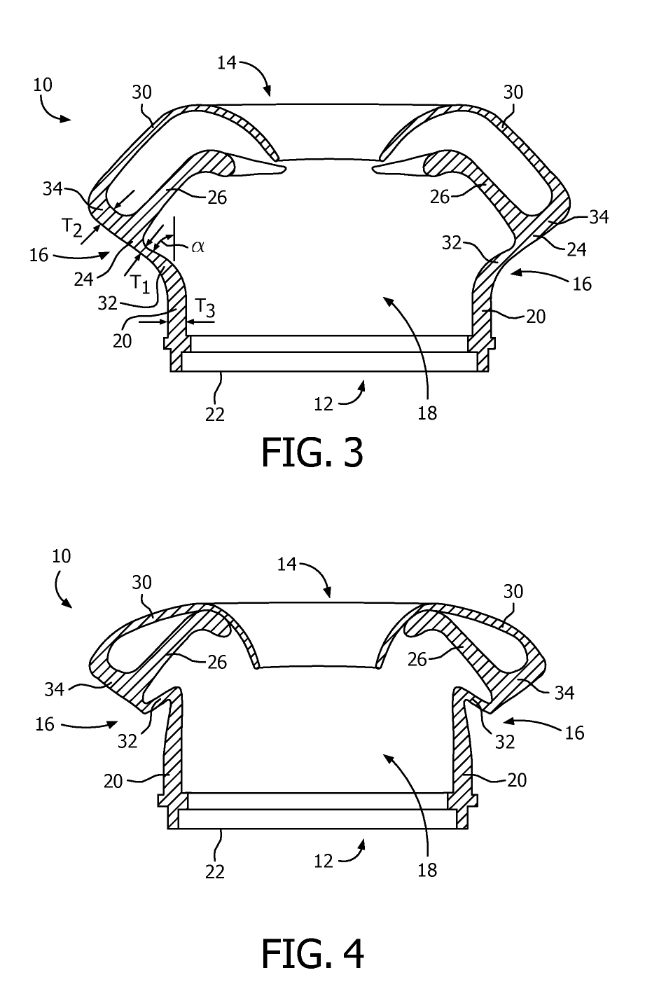

Referring to FIG. 3, angled portion 24 is generally separated into two segments 32, 34 by support portion 26. More particularly, angled portion 24 includes a first segment 32 which extends from base portion 20 to support portion 26, and a second segment 34 which extends from support portion 26 to membrane 30. In the embodiment shown in cross-section in FIGS. 3 and 4, first segment 32 has a predetermined thickness T₁, second segment 34 has a predetermined thickness T₂, and base portion 20 has a predetermined thickness T₃. In the example embodiment illustrated in FIG. 3, T₃=T₂>T₁. In other example embodiments, T₃>T₂>T₁ or T₂>T₃>T₁.

By sizing the thickness of first segment 32 accordingly with respect to the adjoining base portion 20 and second segment 34, first segment 32 generally flexes back toward first portion 12 of cushion 10 as cushion 10 is moved from a relaxed position to a compressed position. Such arrangement acts as a mechanism which generally maintains the lateral positioning of membrane 30 and support portion 26 when moving from an uncompressed position (FIG. 3) to a compressed position (FIG. 4). By maintaining the lateral positioning of membrane 30 and support portion 26 while cushion 10 is moved from a relaxed position to a compressed position, such as when cushion 10 is compressed against the face of a patient, lateral movement of the patient contacting second portion 14 is avoided. Lateral movement of the patient contacting second portion 14 is generally undesirable as such movement tends to uncomfortably distort the patient's face in a manner that may cause one or more of soreness, unwanted marking, and/or cushion leakage.

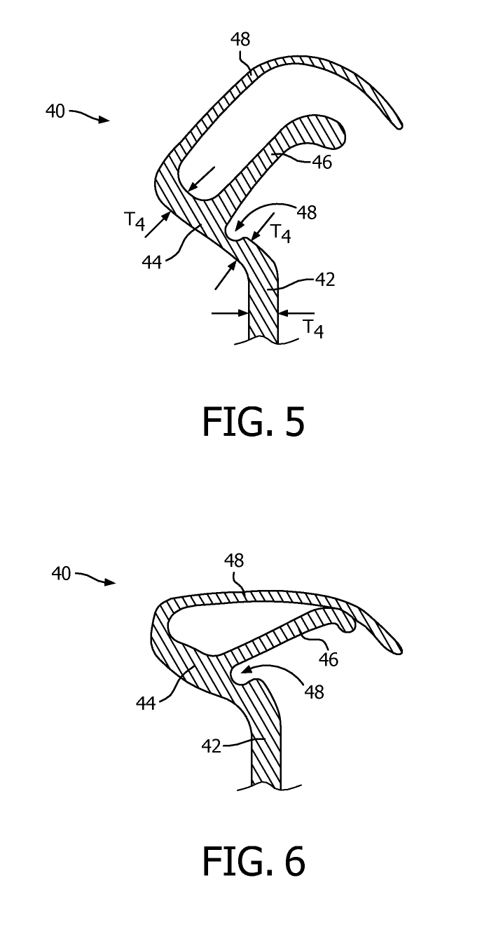

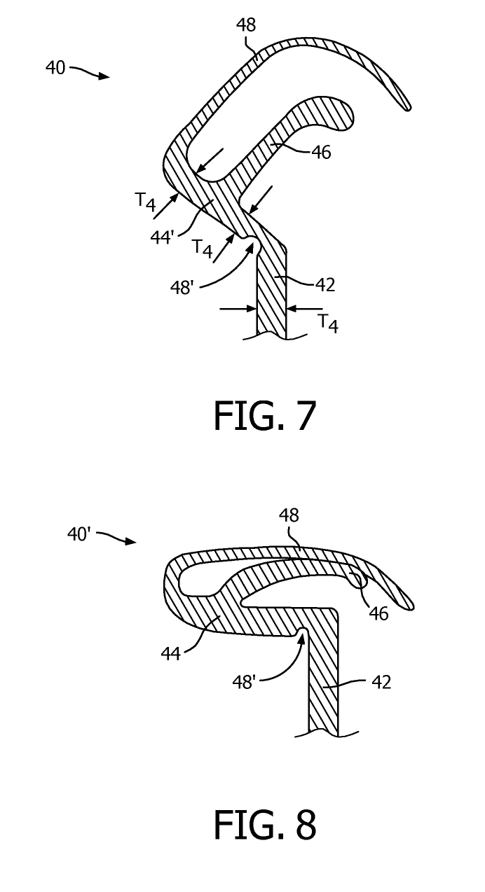

FIGS. 5 and 6 as well as FIGS. 7 and 8 show sectional views of a portion of further cushions 40 and 40′ according to other example embodiments of the present invention. Cushions 40 and 40′, similar to cushion 10 previously discussed, include a base portion 42, an angled portion 44, 44′, a support portion 46 and a membrane 48. However, unlike cushion 10 which utilized various predetermined thicknesses T₁, T₂, and T₃ to provide a mechanism which acts to generally maintain the lateral positioning of membrane 30 and support portion 26 when moving from an uncompressed position to a compressed position, the examples shown in FIGS. 5 and 6 and FIGS. 7 and 8 are of generally the same thickness T₄ and instead utilize a selectively placed notch or groove 48, 48′ to maintain lateral positioning of membrane 48 and support portion 46. More particularly, cushion 40, shown in FIG. 5 (uncompressed) and FIG. 6 (compressed) utilizes groove 48 disposed on an inner side of cushion 40 adjacent support portion 46 to maintain the lateral positioning of membrane 48 and support portion 46. Cushion 40′, shown in FIG. 7 (uncompressed) and FIG. 8 (compressed) utilizes groove 48′ disposed on an outer side of cushion 40′ adjacent base portion 42 to maintain the lateral positioning of membrane 48 and support portion 46.

From the above description it is to be appreciated that embodiments of the present invention provide for a cushion in which the relationship of the patient contacting portions (i.e., the sealing flap and the underlying support structure) maintain their relative positioning when the cushion is moved from an uncompressed to a compressed position. As a result, the width of both the inner and outer portions of the patient contacting side of the cushion maintain generally the same dimensions when a mask utilizing such a cushion is sealingly engaged with the face of a patient. By maintaining such positioning, a cushion constructed according to the present invention provides for a more comfortable and better sealing fit than other cushions presently being used.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion for use in a patient interface device, the cushion comprising:
a first portion structured to be coupled to a frame of a patient interface;
an opposite second portion; and
a wall portion disposed between the first portion and the second portion, the wall portion comprising:
a base portion extending from the first portion; and
an angled portion extending from the base portion generally outward from a centerline of the cushion at an angle (α) with respect to the base portion toward the second portion, wherein the second portion includes:
an underlying support portion which extends inward toward the centerline from about a mid-portion of the angled portion, and
a membrane, structured to sealingly engage a patient's face, which extends inward generally from an end of the angled portion opposite the base portion, and wherein the angled portion is structured to generally maintain the lateral positioning of the membrane and the support portion when the cushion is transitioned from an uncompressed position to a compressed position.

2. The cushion of claim 1, wherein the angled portion is generally separated into a first segmental and a second segment by the support portion, wherein the first segment extends from the base portion to the support portion and the second segment extends from the support portion to the membrane, and wherein the first segment has a predetermined thickness T₁, the second segment has a predetermined thickness T₂ and the base portion has a predetermined thickness T₃; and wherein T₃=T₂>T₁.

3. The cushion of claim 1, wherein the angled portion is generally separated into a first segment and a second segment by the support portion, wherein the first segment extends from the base portion to the support portion and the second segment extends from the support portion to the membrane, and wherein the first segment has a predetermined thickness T₁, the second segment has a predetermined thickness T₂ and the base portion has a predetermined thickness T₃; and wherein T₃>T₂>T₁.

4. The cushion of claim 1, wherein the angled portion is generally separated into a first segment and a second segment by the support portion, wherein the first segment extends from the base portion to the support portion and the second segment extends from the support portion to the membrane, and wherein the first segment has a predetermined thickness T₁, the second segment has a predetermined thickness T₂ and the base portion has a predetermined thickness T₃; and wherein T₂>T₃>T₁.

5. The cushion of claim 1, wherein, the angled portion includes a selectively placed groove which is structured to maintain lateral positioning of the membrane and the support portion when the cushion is transitioned from an uncompressed position to a compressed position.

6. The cushion of claim 5, wherein the groove is disposed adjacent the support portion.

7. The cushion of claim 5, wherein the groove is disposed adjacent the base portion.
